(19) United States
(12) Patent Application Publication
(10) Pub. No.: US 2014/0302131 A1
(43) Pub. Date: Oct. 9, 2014

(54) ORAL FORMULATIONS CONTAINING HYALURONIC ACID FOR SUSTAINED DRUG RELEASE

(71) Applicant: Ole Moeller Dall, Frederiksberg (DK)

(72) Inventor: Ole Moeller Dall, Frederiksberg (DK)

(73) Assignee: Novozymes A/S, Bagsvaerd (DK)

(21) Appl. No.: 14/342,486

(22) PCT Filed: Aug. 31, 2012
(86) PCT No.: PCT/EP2012/066975

§ 371 Date: May 28, 2014

Related U.S. Application Data
(60) Provisional application No. 61/531,199, filed on Sep. 6, 2011.

(30) Foreign Application Priority Data
Sep. 2, 2011 (EP) 11179819.5

(51) Int. Cl. A61K 9/48; A61K 31/135; A61K 9/20

(52) U.S. Cl. CPC A61K 9/4866; A61K 9/205; A61K 31/135
USPC 424/452; 424/465; 514/650; 427/2.14

(57) ABSTRACT

The present invention discloses a tablet or a capsule for oral administration comprising 0.5-0% (w/w) hyaluronic acid or a salt thereof, an active pharmaceutical ingredient (API), and a coating.

### FIELD OF THE INVENTION

The present invention relates to sustained-release oral formulations comprising an active pharmaceutical ingredient, hyaluronic acid or a salt thereof, and a coating. The invention also relates to methods for producing these oral formulations, and to the use of these oral formulations in the manufacture of a medicament for the treatment of various diseases.

### BACKGROUND OF THE INVENTION

Controlled-release (CR) is a mechanism used in tablets or capsules whereby the tablets or the capsules are dissolved slowly releasing a drug over time.

The advantages of sustained-release tablets or capsules are that they can often be taken less frequently than immediate-release formulations of the same drug, and that they keep steadier levels of the drug in the bloodstream.

Today, most time-release drugs are formulated so that the active ingredient is embedded in a matrix of insoluble substance(s) (e.g., cellulose derivates and/or acrylics) in such a way that the dissolving drug must find its way out through the holes in the matrix.

In some CR formulations, the drug dissolves into the matrix, and the matrix physically swells to form a gel, allowing the drug to exit through the outer surface of the gel.

Hyaluronic acid (HA) is a natural and linear carbohydrate polymer belonging to the class of non-sulfated glycosaminoglycans. It is composed of beta-1,3-N-acetyl glucosamine and beta-1,4-glucuronic acid repeating disaccharide units with a molecular weight (MW) up to 6 MDa. HA is present in hyaline cartilage, synovial joint fluid, and skin tissue, both dermis and epidermis.

Due to the unique physical and biological properties of HA (including biocompatibility and biodegradability), HA is employed in a wide range of current and developing applications.

WO 03/061626 discloses an injectable, insertable or implantable drug delivery system that form hydrogels when implanted, injected or inserted comprising hyaluronic acid and a pharmaceutically effective compound.

WO 97/28828 discloses a pharmaceutical composition comprising an effective amount of a controlled release polymer and a proteinaceous interleukin-1 inhibitor.

EP 0913149 discloses a drug composition with a controlled drug release rate comprising a high-molecular substance and/or polyvalent metal ions, hyaluronic acid, and a drug.

US 2005/0031546 discloses an abuse-proofed dosage form comprising an active ingredient and a synthetic or natural polymer with a breaking strength of at least 500 N.

### SUMMARY OF THE INVENTION

We have surprisingly shown that an active pharmaceutical ingredient and hyaluronic acid may be formulated into a slow release tablet or capsule for oral administration so we claim:

A tablet or a capsule comprising
a) 0.5-50% (w/w) hyaluronic acid or a salt thereof,
b) an active pharmaceutical ingredient (API), and
c) a coating
   for use in oral administration.

The present invention also claims a method of producing a tablet or a capsule comprising
a) mixing hyaluronic acid or a salt thereof with an active pharmaceutical ingredient (API);
b) granulating the mixture from step a) into pellets;
c) compressing the pellets into a tablet or filling the pellets into a capsule; and
d) coating the tablet or the capsule.

The present invention also claims a method of producing a capsule comprising
a) mixing hyaluronic acid or a salt thereof with an active pharmaceutical ingredient (API);
b) granulating the mixture from step a) into pellets;
c) coating the pellets; and
d) filling the pellets into a capsule.

The present invention also claims a tablet or a capsule comprising
a) 0.5-50% (w/w) hyaluronic acid or a salt thereof,
b) an active pharmaceutical ingredient (API), and
c) a coating,
   for the manufacture of a medicament for use in the treatment of pain, migraine, epilepsy, anxious disorders, inflammatory conditions, infectious diseases, hormonal disorders, cardiovascular diseases, gastro intestinal illnesses or cancer.

### DETAILED DESCRIPTION OF THE INVENTION

The present invention discloses a tablet or a capsule for oral administration comprising 0.5-50% (w/w) hyaluronic acid or a salt thereof, an active pharmaceutical ingredient (API), and a coating.

HA's role in this invention is to increase the viscosity and/or to form a high viscous gel-like core in the tablet or the capsule in order to slow down the release of the API.

It is known in the art that it contributes to a better compliance if a drug can be dosed, e.g., 1-2 times per day instead of 3-4 times per day.

“Hyaluronic acid” is defined herein as an unsulphated glycosaminoglycan composed of repeating disaccharide units of N-acetylglucosamine (GlcNAc) and glucuronic acid (GlcUA) linked together by alternating beta-1,4 and beta-1,3 glycosidic bonds. Hyaluronic acid is also known as hyaluronan, hyaluronate, or HA. The terms hyaluronan, hyaluronic acid, and HA are used interchangeably herein.

The level of hyaluronic acid may be determined according to the modified carbazole method (Bitter and Muir, 1962, Anal Biochem. 4: 330-334).

In a preferred embodiment the hyaluronic acid used according to the invention is of a very pure quality, in particular cGMP.

### HA Sources

Rooster combs are a significant commercial source for hyaluronan. Microorganisms are an alternative source. U.S. Pat. No. 4,801,539 and EP 0694616 disclose fermentation methods for preparing hyaluronic acid involving a strain of Streptococcus zooepidemicus.

In a preferred embodiment the hyaluronic acid or salt thereof is of microbial origin, preferably recombinantly produced. In a preferred embodiment the hyaluronic acid or salt thereof is produced by a Gram-positive bacterium such as Bacillus.

In a most preferred embodiment the hyaluronic acid or salt thereof is produced by Bacillus subtilis. The hyaluronic acid or salt thereof may be produced as disclosed in WO 03/054163.

Hyaluronan synthases have been described from vertebrates, bacterial pathogens, and algal viruses (DeAngelis, P. L., 1999, Cell. Mol. Life Sci. 56: 670-682). WO 99/23227 discloses a Group I hyaluronate synthase from Streptococcus equisimilis. WO 99/51265 and WO 00/27437 describe a Group II hyaluronate synthase from Pasturella multocida. Ferretti et al. disclose the hyaluronan synthase operon of Streptococcus pyogenes, which is composed of three genes, hasA, hasB, and hasC, that encode hyaluronate synthase, UDP glucose dehydrogenase, and UDP-glucose pyrophosphorylase, respectively (Proc. Natl. Acad. Sci. USA. 98, 4658-4663, 2001). WO 99/51265 describes a nucleic acid segment having a coding region for a Streptococcus equisimilis hyaluronan synthase.

The host cell may be any Bacillus cell suitable for recombinant production of hyaluronic acid. The Bacillus host cell may be a wild-type Bacillus cell or a mutant thereof. Bacillus cells useful in the practice of the present invention include, but are not limited to, Bacillus agaraderhens, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, land Bacillus thuringiensis cells. Mutant Bacillus subtilis cells particularly adapted for recombinant expression are described in WO 98/22598. Non-encapsulating Bacillus cells are particularly useful in the present invention.

Since the hyaluronan of a recombinant Bacillus cell is expressed directly to the culture medium, a simple process may be used to isolate the hyaluronan from the culture medium. First, the Bacillus cells and cellular debris are physically removed from the culture medium. The culture medium may be diluted first, if desired, to reduce the viscosity of the medium. Many methods are known to those skilled in the art for removing cells from culture medium, such as centrifugation or microfiltration. The remaining supernatant may then be filtered, such as by ultrafiltration, to concentrate and remove small molecule contaminants from the hyaluronan. Various purification steps may then be performed depending on the required purity of the final product. The hyaluronan may be dried or concentrated by using evaporative techniques known to the person skilled in the art, such as lyophilization or spray drying.

### Salts of Hyaluronic Acid

Any salt of hyaluronic acid may be used according to the present invention.

In a preferred embodiment the salt of hyaluronic acid is an inorganic salt, preferably sodium hyaluronate, potassium hyaluronate, ammonium hyaluronate, calcium hyaluronate, magnesium hyaluronate, zinc hyaluronate, or cobalt hyaluronate.

### HA Molecular Weight

Any molecular weight may be used according to the invention but in a preferred embodiment the hyaluronic acid or the salt thereof has an average molecular weight in the range of 200-6000 kDa; more preferably an average molecular weight in the range of 300-6000 kDa; more preferably an average molecular weight in the range of 400-6000 kDa; and even more preferably an average molecular weight in the range of 500-6000 kDa. The molecular weight may be determined as known in the art.

### Active Pharmaceutical Ingredient (API)

The active pharmaceutical ingredient of interest may be any compound known in the art suitable for oral administration, e.g., a peptide, a protein, an enzyme, a hormone, an antibiotic, a fungicide or an analgesic.

In a preferred embodiment the active pharmaceutical ingredient is an analgesic (also known as a painkiller). An analgesic is any member of the group of drugs used to relieve pain (achieve analgesia).

Analgesic drugs act in various ways on the peripheral and central nervous systems; they include paracetamol (para-acetylaminophenol), the non-steroidal anti-inflammatory drugs (NSAIDs such as COX-1 and COX-2, selective and non-selective), and opioid drugs. The opioid drugs may be of natural or synthetic origin, and analogs are included, e.g., morphine and opium.

According to the present invention an analgesic selected from the group consisting of paracetamol, non-steroidal anti-inflammatory drugs, and opioid drugs are preferred.

An example of a useful analgesic is Tramadol hydrochloride. Tramadol hydrochloride is a centrally-acting analgesic, used in treating moderate to moderately severe pain. It is a synthetic analog of the phenanthrene alkaloid codeine and, as such, it is an opioid drug and also a pro-drug (codeine is metabolized to morphine in the body). Tramadol has a wide range of applications, including treatment for restless leg syndrome, acid reflux, and fibromyalgia.

### Tablets or Capsules

A tablet is a pharmaceutical dosage form. It comprises a mixture of active substance(s) and one or more excipients, usually in powder form, pressed or compacted from a granule into a solid dose.

The compressed tablet is the most popular dosage form in use today. About two-thirds of all prescriptions are dispensed as solid dosage forms, and half of these are compressed tablets. A tablet can be formulated to deliver an accurate dosage to a specific site, and it is usually taken orally.

Medicinal tablets were originally made in the shape of a disk of whatever color their components determined, but are now made in many shapes and colors to help distinguish different medicines. Tablets are often embossed (or printed on outer coating) with symbols, letters, and numbers, which enable them to be identified. The size of a tablet to be swallowed may range from a few millimeters to about a centimeter.

Some tablets are in the shape of capsules. Medicinal tablets and capsules are often called pills. This is technically incorrect, since tablets are made by compression, whereas pills are ancient solid dose forms prepared by rolling a soft mass into a round shape.

### Manufacture of the Tableting Blend

According to the present invention the tablets and the capsules are produced as known in the art. The tablet or the capsule are for oral administration and comprises
a) 0.5-50% (w/w) hyaluronic acid or a salt thereof,
b) an active pharmaceutical ingredient (API), and
c) a coating.

In a preferred embodiment the tablet or the capsule comprises 0.5-49% (w/w) hyaluronic acid or a salt thereof, in particular the tablet or the capsule comprises 0.5-48% (w/w) hyaluronic acid or a salt thereof, in particular the tablet or the capsule comprises 0.5-47% (w/w) hyaluronic acid or a salt thereof, in particular the tablet or the capsule comprises 0.5-46% (w/w) hyaluronic acid or a salt thereof, in particular the tablet or the capsule comprises 0.5-45% (w/w) hyaluronic acid or a salt thereof, in particular the tablet or the capsule comprises 0.5-44% (w/w) hyaluronic acid or a salt thereof, in particular the tablet or the capsule comprises 0.5-43% (w/w) hyaluronic acid or a salt thereof, in particular the tablet or the capsule comprises 0.5-42% (w/w) hyaluronic acid or a salt thereof, in particular the tablet or the capsule comprises 0.5-41% (w/w) hyaluronic acid or a salt thereof, in particular the tablet or the capsule comprises 0.5-40% (w/w) hyaluronic acid or a salt thereof, in particular the tablet or the capsule comprises 0.5-39% (w/w) hyaluronic acid or a salt thereof, in particular the tablet or the capsule comprises 0.5-38% (w/w) hyaluronic acid or a salt thereof, in particular the tablet or the capsule comprises 0.5-37% (w/w) hyaluronic acid or a salt thereof, in particular the tablet or the capsule comprises 0.5-36% (w/w) hyaluronic acid or a salt thereof, in particular the tablet or the capsule comprises 0.5-35% (w/w) hyaluronic acid or a salt thereof, in particular the tablet or the capsule comprises 0.5-34% (w/w) hyaluronic acid or a salt thereof, in particular the tablet or the capsule comprises 0.5-33% (w/w) hyaluronic acid or a salt thereof, in particular the tablet or the capsule comprises 0.5-32% (w/w) hyaluronic acid or a salt thereof, in particular the tablet or the capsule comprises 0.5-31% (w/w) hyaluronic acid or a salt thereof, in particular the tablet or the capsule comprises 0.5-30% (w/w) hyaluronic acid or a salt thereof, in particular the tablet or the capsule comprises 0.5-29% (w/w) hyaluronic acid or a salt thereof, in particular the tablet or the capsule comprises 0.5-28% (w/w) hyaluronic acid or a salt thereof, in particular the tablet or the capsule comprises 0.5-27% (w/w) hyaluronic acid or a salt thereof, in particular the tablet or the capsule comprises 0.5-26% (w/w) hyaluronic acid or a salt thereof, in particular the tablet or the capsule comprises 0.5-25% (w/w) hyaluronic acid or a salt thereof, in particular the tablet or the capsule comprises 0.5-24% (w/w) hyaluronic acid or a salt thereof, in particular the tablet or the capsule comprises 0.5-23% (w/w) hyaluronic acid or a salt thereof, in particular the tablet or the capsule comprises 0.5-22% (w/w) hyaluronic acid or a salt thereof, in particular the tablet or the capsule comprises 0.5-21% (w/w) hyaluronic acid or a salt thereof, in particular the tablet or the capsule comprises 0.5-20% (w/w) hyaluronic acid or a salt thereof, in particular the tablet or the capsule comprises 0.5-19% (w/w) hyaluronic acid or a salt thereof, in particular the tablet or the capsule comprises 0.5-18% (w/w) hyaluronic acid or a salt thereof, in particular the tablet or the capsule comprises 0.5-17% (w/w) hyaluronic acid or a salt thereof, in particular the tablet or the capsule comprises 0.5-16% (w/w) hyaluronic acid or a salt thereof, in particular the tablet or the capsule comprises 0.5-15% (w/w) hyaluronic acid or a salt thereof, in particular the tablet or the capsule comprises 0.5-14% (w/w) hyaluronic acid or a salt thereof, in particular the tablet or the capsule comprises 0.5-13% (w/w) hyaluronic acid or a salt thereof, in particular the tablet or the capsule comprises 0.5-12% (w/w) hyaluronic acid or a salt thereof, in particular the tablet or the capsule comprises 0.5-11% (w/w) hyaluronic acid or a salt thereof, in particular the tablet or the capsule comprises 0.5-10% (w/w) hyaluronic acid or a salt thereof, in particular the tablet or the capsule comprises 0.5-9.0% (w/w) hyaluronic acid or a salt thereof, in particular the tablet or the capsule comprises 0.5-8.0% (w/w) hyaluronic acid or a salt thereof, in particular the tablet or the capsule comprises 0.5-7.0% (w/w) hyaluronic acid or a salt thereof, in particular the tablet or the capsule comprises 0.5-6.0% (w/w) hyaluronic acid or a salt thereof, and especially the tablet or the capsule comprises 0.5-5.0% (w/w) hyaluronic acid or a salt thereof.

In the tablet pressing process, the main guideline is to ensure that the appropriate amount of an active ingredient is placed in each tablet.

Hence, all the ingredients should be well-mixed. If a sufficiently homogenous mix of the components cannot be obtained with simple blending processes, the ingredients may be, e.g., granulated prior to compression to assure an even distribution of the active compound in the final tablet.

Two basic techniques are used to granulate powders for compression into a tablet: wet granulation and dry granulation. Powders that can be mixed well do not require granulation and can be compressed into tablets through direct compression.

### Wet Granulation

Wet granulation is a process of using a liquid binder to lightly agglomerate the powder mixture. The amount of liquid has to be properly controlled, as over-wetting will cause the granules to be too hard and under-wetting will cause them to be too soft and friable. A typical procedure for wet granulation has the following steps:
Step 1: The active ingredient and excipient(s) are weighed and mixed.
Step 2: The wet granulate is prepared by adding the liquid binder/adhesive to the powder blend and mixing thoroughly. Examples of binders/adhesives include aqueous preparations of corn starch, natural gums such as acacia, cellulose derivatives such as methyl cellulose, gelatin, and povidone.
Step 3: Sieving the damp mass through a mesh to form granules.
Step 4: Drying the granules. A conventional tray-dryer or fluid-bed dryer are most commonly used.
Step 5: After the granules are dried, they are passed through a screen of smaller/uniform size than the one used for the wet mass in order to create granules of uniform size.

Low shear wet granulation processes use very simple mixing equipment, and can take a considerable time to achieve a uniformly mixed state. High shear wet granulation processes use equipment that mixes the powder and liquid at a very fast rate, and thus speeds up the manufacturing process. Fluid bed granulation is a multiple-step wet granulation process performed in the same vessel to pre-heat, granulate, and dry the powders. It is used because it allows close control of the granulation process.

### Dry Granulation

Dry granulation processes create granules by light compaction of the powder blend under low pressures. The compacts so-formed are broken up gently to produce granules (agglomerates).

The dry granulation process is often used when the product to be granulated is sensitive to moisture and/or heat. Dry granulation can be conducted on a tablet press using slugging tooling or on a roll press called a roller compactor.

Dry granulation equipment offers a wide range of pressures to attain proper densification and granule formation. Dry granulation is simpler than wet granulation, therefore the cost is reduced. However, dry granulation often produces a higher percentage of fine granules, which can compromise the quality or create yield problems for the tablet.

Dry granulation requires drugs or excipients with cohesive properties, and a ‘dry binder’ may need to be added to the formulation to facilitate the formation of granules.

### Granule Lubrication

After granulation, a final lubrication step is normally used to ensure that the tableting blend does not stick to the equipment during the tableting process. This usually involves low shear blending of the granules with a powdered lubricant, such as magnesium stearate, talcum, stearic acid or silicon dioxide.

### Additional Excipients

The additional excipients may include diluents, binders or granulating agents, glidants (flow aids) and lubricants to ensure efficient tabletting; disintegrants to promote tablet break-up in the digestive tract; sweeteners or flavours to enhance taste; and pigments to make the tablets visually attractive.

According to the present invention a preferred additional excipient is selected from the group consisting of a monosaccharide, a disaccharide, an oligosaccharide, a polysaccharide, sugar alcohol, and talcum; in particular lactose, and cellulose, especially microcrystalline cellulose.

### Coating

The tablet/capsule coatings must be stable and strong enough to survive the handling of the tablet/capsule, must not make tablets/capsules stick together during the coating process, and must follow the fine contours of embossed characters or logos on tablets.

A coating is also applied to make the tablet or the capsule smoother and easier to swallow, to control the release rate of the active ingredient, and/or to make the tablet or the capsule more resistant to the environment (e.g. gastric resistance). Tablet/capsule coatings are also useful to extend the shelf-life of components that are sensitive to moisture or oxidation.

According to the present invention the coating is especially applied in order to make the tablet or capsule more gastric resistant compared to a tablet or a capsule without a coating. Gastric resistant (or enteric resistant) mean that the coating is resistant to stomach acid, but dissolves in the less acidic area of the intestines or colon.

In a preferred embodiment the coating is a polymer. In a preferred embodiment the polymer has gastro resistant (acid resistant) properties. In a preferred embodiment the polymer is a cellulose derivate such as as a hypromelose or an acrylate, in particular methacrylic acid or methyl methacrylate.

### Dissolution Studies

One way of showing that the active pharmaceutical compound is released slower in the digestive system/gastro intestinal tract—compared to a tablet or a capsule without the hyaluronic acid or the salt thereof—is by using dissolution studies.

Dissolution studies are a very used tool in the characterization of solid controlled release formulations. Dissolution is the process by which a solid, semi solid or liquid API enters into a solution in the presence of solvents.

Dissolution rate may be defined as the amount of active ingredient in a dosage form dissolved in unit per time under standardized conditions of agitation/flow, temperature, and media composition. The conditions used in standard testing are described both in European Pharmacopoeia (6.2) and USP (NF29 General Chapters <711> DISSOLUTION).

Among the different in vitro systems available for semi-solid testing, the flow through system (USP Method 4) offers the best characteristics. It has been employed for many years in the testing of different dosage forms such as tablets, capsules and gels. It is the method of choice for extended release and for poorly soluble products. The method is used in Example 1.

### Utility and Advantages of the Invention

The use of a tablet or a capsule according to the present invention may be for the manufacture of a medicament for the treatment of pain, migraine, epilepsy, anxious disorders, inflammatory conditions, infectious diseases, hormonal disorders, cardiovascular diseases, gastro intestinal illnesses or cancer. Relevant illnesses and conditions are:
Pain (acute pains, cancer pains, chronic pains)
Migraine (acute and preventive treatment)
Epilepsy (preventive)
Anxious disorders (long term treatments)
Inflammatory conditions (acute and preventive treatment with and against steroids, NSAID's, rheumatism, psoriasis, osteoporosis and other autoimmune illnesses)
Infectious disease (antibiotics and fungicides)
Hormonal disorders (deficiency diseases)
Contraceptives
Cardiovascular diseases (mostly preventive and maintenance dose)
Gastro intestinal illnesses (acute and preventive treatment)
Oral chemotherapy and oral cancer drugs.

The tablet or the capsule may particularly be a medicament for use in the treatment of pain according to the present invention.

The tablet or the capsule may particularly be a medicament for use in the treatment of migraine according to the present invention.

The tablet or the capsule may particularly be a medicament for use in the treatment of epilepsy according to the present invention.

The tablet or the capsule may particularly be a medicament for use in the treatment of an anxious disorder according to the present invention.

The tablet or the capsule may particularly be a medicament for use in the treatment of inflammatory conditions according to the present invention.

The tablet or the capsule may particularly be a medicament for use in the treatment of an infectious disease according to the present invention.

The tablet or the capsule may particularly be a medicament for use in the treatment of a hormonal disorder according to the present invention.

The tablet or the capsule may particularly be a medicament for use in the treatment of a cardiovascular disease according to the present invention.

The tablet or the capsule may particularly be a medicament for use in the treatment of a gastro intestinal illness according to the present invention.

The tablet or the capsule may particularly be a medicament for use in the treatment of cancer according to the present invention.

The invention is further illustrated in the following example which is not intended to be in any way limiting to the scope of the invention as claimed.

### EXAMPLES

### Example 1

Release of Tramadol Hydrochloride from Formulated Tablets Containing Hyaluronic Acid

The purpose of this experiment was to investigate the use of HA as a controlled release (CR) agent in tablet formulations for oral applications. The release of the API, tramadol hydrochloride, was evaluated through dissolution studies. The dissolution profile of tramadol hydrochloride in a commercial CR tablet formulation was compared to that in tablet formulations containing HA at various concentrations.

CR tablet Nobligan® Retard was evaluated and compared with a tablet formulation containing HA at different concentrations. The compositions of the formulations are shown in tables 1a and 1b.

TABLE 1a

| | | |
|---|---|---|
| Tramadol hydrochloride | Active | 100 |
| Microcrystalline cellulose | Filler | N/A |
| Hydroxypropyl methylcellulose | Binder | N/A |
| Silicon dioxide | Lubricant | N/A |
| Magnesium stearate | Lubricant | N/A |

TABLE 1b

| | | Concentration, |
|---|---|---|
| Compound | Function | mg/tablet |
| Tramadol hydrochloride | Active | 100.0 |
| Microcrystalline cellulose | Filler | 210.0 |
| Lactose anhydrous | Filler | 50.0 |
| Polyvinylpyrrolidone | Binder | 22.0 |
| Hydroxypropyl cellulose | Binder | 8.0 |
| HA (850 kDa) | Controlled release agent | 4.0-16.0 |
| Silicon dioxide | Lubricant | 4.0 |
| Magnesium stearate | Lubricant | 2.0 |

### Gastro Resistant Coating

In order to protect both the API and HA in the tablet from the acidic environment in the stomach (˜pH 1-2), and to secure release of the API in the pH neutral small intestine (˜pH 6-8) the tablet may be coated with an acid resistant coating.

The coating may consist of a methyl acrylate, methyl methacrylate and methacrylic acid (Eudragit® FS 30 D). This coating is insoluble in acidic media, but dissolves by salt formation above pH 7.0. It is a milky-white liquid of low viscosity with a faint characteristic odor in the aqueous dispersion of an anionic copolymer: Poly(methyl acrylate-co-methyl methacrylate-co-methacrylic acid).

The coating may be performed in a fluid bed coater with top spray.

TABLE 1c
Coating process settings

| Parameter | Setting | |
|---|---|---|
| Inlet air temperature | 35-45° | C. |
| Outlet air temperature | 25-30° | C. |
| Product temperature | 25-28° | C. |
| Drying air volume | 50-90 | m³/h |
| Nozzle pressure (atomizing air) | 1.0-2.0 | bar |
| Spray rate | 10-15 | g/min/kg |
| Drying temp. (final) | 40° | C. |

TABLE 1d
Coating materials

| Compound | Amount |
|---|---|
| Tramadol hydrochloride/hyaluronic acid tablet cores | 300 g |
| Eudragit ® FS 30 D, Aqueous Dispersion 30% | 50 g |
| Purified water (solvent) | 30 g |

The coating layer on the dried tablets will consist of approx. 15 mg per tablet.

### Analytical Methods

The test system was based on the flow through system (USP Method 4): A flow-through cell (sample cell) where a test sample is placed in a specially designed cell through which media is pumped at a set and controlled temperature. The media is filtered upon leaving the cell and is then analyzed by UV. The type of cell is chosen to suit the dosage form, and it is permanently maintained at the set temperature (37° C.) in a water jacket.

### Prepared Formulations:

Formulations to be tested in the dissolution system are listed in Table 2.

TABLE 2

| Formulation | % HA added |
|---|---|
| Tramadol hydrochloride | 0 |
| powder (control) 100 mg | |
| Tradolan ® 50 mg tablets | 0 (commercial product from Lannacher) |
| Nobligan ® Retard | 0 (commercial product from Grünenthal) |
| 100 mg tablets | |
| HA 0.0 100 mg tablets | 0 (control) |
| HA 0.5 100 mg tablets | 0.5 |
| HA 1.0 100 mg tablets | 1.0 |
| HA 1.5 100 mg tablets | 1.5 |
| HA 2.0 100 mg tablets | 2.0 |
| HA 4.0 100 mg tablets | 4.0 |

The dissolution system was prepared with the settings displayed in Table 3a, and every single cell was loaded with 1 tablet. Cell number 7 was blank and loaded with buffer.

TABLE 3a
(Parameters and settings for the dissolution instrument)

| Parameter | Setting |
|---|---|
| Number of cells per analysis | 6 + 1 blank |
| Cell type | 22.4 mm cell |
| Amount of glass beads | 1 measuring spoon |
| Volume of media per cell | 250.0 mL |
| Media | Phosphate buffer pH 6.8 |
| Filter | 1 glass fiber filter + 0.1 μm membrane |
| | filter + 2 glass fiber filters |
| Flow rate | 8 mL/min |
| Temperature | 37° C. |
| Total dosing of API | 100 mg |
| Total amount of formulation | 1 tablet per cell |
| Position of formulation in cell | On top of glass beads |
| Stirring speed | 7-9 |
| UV detection | 270 nm |

Because the tested tablets are CR formulations which are designed to give release of the API in the intestinal system, a phosphate buffer pH 6.8 is used as dissolution media to simulate human intestinal fluids.

In order to demonstrate the gastro resistant properties of the tablet coating, the tablet may be exposed to 0.1 M hydrochloric acid for up to 2 hours in the dissolution instrument. Afterwards the media may be changed to a 0.2 M Phosphate buffer at pH 7.8. This media change will happen automatically.

TABLE 3b
Testing of gastro resistant tablets (Parameters and settings
for the dissolution instrument)

| Parameter | Setting |
|---|---|
| Number of cells per analysis | 6 + 1 blank |
| Cell type | 22.4 mm cell |
| Amount of glass beads | 1 measuring spoon |
| Volume of media per cell | 250.0 mL |
| Media | |
| 0-2 hours | Media 1: 0.1M Hydrochloric acid pH 1 |
| 2 hours and forward | Media 2: 0.2M Phosphate buffer pH 7.8 |
| Filter | 1 glass fiber filter + 0.1 μm membrane |
| | filter + 2 glass fiber filters |
| Flow rate | 8 mL/min |
| Temperature | 37° C. |
| Total dosing of API | 100 mg |
| Total amount of formulation | 1 tablet per cell |
| Position of formulation in cell | On top of glass beads |
| Stirring speed | 7-9 |
| UV detection | 270 nm |

### RESULTS

The results from the dissolution analyses (Table 3a) are summarized in Tables 4 and 5.

TABLE 4
(Release times of tramadol hydrochloride from formulated tablets in 20 mM
phosphate buffer)

| | Time, | | | | | | | | | Time, |
|---|---|---|---|---|---|---|---|---|---|---|
| Tablet | minutes | | | | | | | | | hours |
| sample | Cell 1 | Cell 2 | Cell 3 | Cell 4 | Cell 5 | Cell 6 | average | s | % RSD | (Average) |
| HA 0.0% | 130 | 125 | 125 | 125 | 130 | 125 | 130 | 2.58 | 2.0 | 2.2 |
| HA 0.5% | 160 | 165 | 160 | 155 | 155 | 160 | 159 | 3.76 | 2.4 | 2.7 |
| HA 1.0% | 190 | 190 | 190 | 195 | 185 | 185 | 189 | 3.76 | 2.0 | 3.2 |
| HA 1.5% | 220 | 225 | 225 | 225 | 220 | 230 | 224 | 3.76 | 1.7 | 3.7 |
| HA 2.0% | 270 | 270 | 290 | 270 | 290 | 280 | 278 | 9.83 | 3.5 | 4.6 |
| HA 4.0% | 410 | 390 | 390 | 400 | 400 | 410 | 400 | 8.94 | 2.2 | 6.7 |

The release of 100 mg of tramadol hydrochloride from formulated tablets with HA in concentration from 0% to 4% is shown in Table 4.

Overall, the results show that the release time of the API increases with the HA concentration. For instance, 100% of the API is released after approx. 2.7 hours from tablets with 0.5% HA, and after approx.4.6 hours from tablets with 2.0% HA, while it takes approx. 6.7 hours to reach a 100% release from tablets with 4.0% HA. The results show a prolonged release time of API when using increasing amounts of HA.

TABLE 5
Release times of tramadol hydrochloride from formulated tablets and from the commercial
tablets (Tradolan ® and Nobligan ®) in 200 mM phosphate buffer

| | Time, | | | | | | | | | Time, |
|---|---|---|---|---|---|---|---|---|---|---|
| Tablet | minutes | | | | | | | | | hours |
| Sample | Cell 1 | Cell 2 | Cell 3 | Cell 4 | Cell 5 | Cell 6 | average | s | % RSD | (Average) |
| HA 0% | 155 | 155 | 160 | 155 | 160 | 155 | 157 | 2.58 | 1.6 | 2.6 |
| HA 0.5% | 195 | 200 | 195 | 190 | 200 | 200 | 197 | 4.08 | 2.1 | 3.3 |
| HA 1.0% | 215 | 225 | 220 | 225 | 220 | 230 | 223 | 5.24 | 2.4 | 3.7 |
| HA 1.5% | 300 | 300 | 300 | 310 | 300 | 290 | 300 | 6.32 | 2.1 | 5.0 |
| HA 2.0% | 440 | 420 | 410 | 410 | 420 | 440 | 423 | 1.66 | 3.2 | 7.1 |
| HA 4.0% | 610 | 630 | 600 | 600 | 600 | 630 | 612 | 14.72 | 2.4 | 10.2 |
| Tradolan | 35 | 40 | 35 | 40 | 35 | 40 | 38 | 2.74 | 7.3 | 0.6 |
| Nobligan | 1140 | 1060 | 1120 | 1100 | 1120 | 1100 | 1107 | 27.33 | 2.5 | 18.4 |

The release of 100 mg of tramadol hydrochloride from formulated tablets with HA in concentration from 0% to 4% is shown in Table 5. The table illustrates that 100% of the API is released after approx. 3.3 hours from tablets with 0.5% HA, after approx. 7.1 hours from tablets with 2.0% HA and after approx. 10.2 hours from tablets with 4.0% HA. The results show a prolonged release time of API when using increasing amounts of HA.

The results also show a prolonged release time of API when using 200 mM phosphate buffer instead of 20 mM phosphate buffer (approx. 10 hours compared of approx. 7 hours from tablets with 4.0% HA).

The tested formulations with HA from 0.5% to 2.0% can be characterized as medium slow release formulations because of the clear delay in API release. The tested 4.0% HA formulation can be characterized as a slow release formulation.

The tablets containing HA were compared with commercial tablets (Tradolan® and Nobligan®) in their performance to release tramadol hydrochloride (see Table 5).

The complete release from Tradolan® is observed after 0.6 hours and from Nobligan® after approx 18.4 hours.

The results show that the formulated tablets have a higher release time compared to the fast release commercial tablet Tradolan®.

The tablets with 4% HA and Nobligan® are slow release tablets, but the release time of API from Nobligan® is higher than the release time from the prepared tablets.

### CONCLUSION

We have shown in Example 1 that HA is a slow release agent in a solid dose formulation.

0.5-4.0% HA in the formulated tablets has a very significant effect on the release profile of API (tramadol hydrochloride) compared to the tablet without HA. This indicates that the formulation can be changed from a medium release formulation to a slow release formulation by adding HA.

1. A tablet or a capsule comprising
a) 0.5-50% (w/w) hyaluronic acid or a salt thereof,
b) an active pharmaceutical ingredient (API), and
c) a coating
for use in oral administration.

2. The tablet or the capsule according to claim 1, wherein the salt of hyaluronic acid is selected from the group consisting of sodium hyaluronate, potassium hyaluronate, ammonium hyaluronate, calcium hyaluronate, magnesium hyaluronate, zinc hyaluronate, and cobalt hyaluronate.

3. The tablet or the capsule according to claim 1, wherein the active pharmaceutical ingredient (API) is a peptide, a protein, an enzyme, a hormone, an antibiotic, a fungicide or an analgesic.

4. The tablet or the capsule according to claim 1, wherein the coating is a polymer.

5. The tablet or the capsule according to claim 1, wherein the tablet or the capsule comprises an additional excipient.

6. The tablet or the capsule according to claim 5, wherein the additional excipient is selected from the group consisting of a monosaccharide, a disaccharide, an oligosaccharide, a polysaccharide, a sugar alcohol and talcum.

7. The tablet or the capsule according to claim 6, wherein the additional excipient is lactose or cellulose.

8. The tablet or the capsule according to claim 1, wherein the active pharmaceutical compound is released slower in the gastro intestinal tract compared to a tablet or a capsule without the hyaluronic acid or the salt thereof.

9. A method of producing a tablet or a capsule according to claim 1 comprising
a) mixing hyaluronic acid or a salt thereof with an active pharmaceutical ingredient (API);
b) granulating the mixture from step a) into pellets;
c) compressing the pellets into a tablet or filling the pellets into a capsule; and
d) coating the tablet or the capsule.

10. A method of producing a capsule according to claim 1 comprising
a) mixing hyaluronic acid or a salt thereof with an active pharmaceutical ingredient (API);
b) granulating the mixture from step a) into pellets;
c) coating the pellets; and
d) filling the pellets into a capsule.

11. A tablet or a capsule as defined in claim 1 for use in the manufacture of a medicament for the treatment of pain, migraine, epilepsy, anxious disorders, inflammatory conditions, infectious diseases, hormonal disorders, cardiovascular diseases, gastro intestinal illnesses or cancer.

12. The tablet or the capsule according to claim 11, wherein the medicament is for the treatment of pain.
